(19) United States
(12) Patent Application Publication
(10) Pub. No.: US 2026/0070861 A1
(43) Pub. Date: Mar. 12, 2026

(54) PREPARATION METHOD OF AROMATIC HYDROCARBON OLIGOMER

(71) Applicant: CHINA UNIVERSITY OF PETROLEUM—BEIJING, Beijing (CN)

(72) Inventors: Zhichang LIU, Beijing (CN); Mengmeng XU, Beijing (CN); Tao ZHENG, Beijing (CN); Haowei LIU, Beijing (CN); Tianli HUI, Beijing (CN); Rui ZHANG, Beijing (CN); Xianghai MENG, Beijing (CN); Haiyan LIU, Beijing (CN)

(73) Assignee: CHINA UNIVERSITY OF PETROLEUM—BEIJING, Beijing (CN)

(21) Appl. No.: 19/391,955

(22) Filed: Nov. 17, 2025

Related U.S. Application Data
(63) Continuation of application No. PCT/CN2024/125069, filed on Oct. 15, 2024.

(30) Foreign Application Priority Data
Dec. 18, 2023 (CN) 202311744989.2
Jan. 17, 2024 (CN) 202410070565.0

(51) Int. Cl. C07C 2/66

(52) U.S. Cl. CPC C07C 2/66

(57) ABSTRACT

The present application provides a preparation method of an aromatic hydrocarbon oligomer, the preparation method including: subjecting an aromatic hydrocarbon compound to an oligomerization reaction under the catalysis of a chloroaluminate ionic liquid to obtain the aromatic hydrocarbon oligomer, where the oligomerization reaction is carried out in a solvent system, and the solvent is selected from chlorinated hydrocarbon solvents, and the aromatic hydrocarbon compound is selected from fused-ring aromatic hydrocarbons with 2 to 4 rings. The preparation method of the present application can efficiently prepare aromatic hydrocarbon oligomers with simple composition and narrow molecular weight distribution under mild conditions, thereby providing a high-quality precursor for the preparation of high-quality mesophase pitch.

### CROSS-REFERENCE TO RELATED APPLICATION

This application is a continuation application of PCT application serial no. PCT/CN2024/125069, filed on Oct. 15, 2024, which claims the priority benefit of China application no. 202311744989.2, filed on Dec. 18, 2023, and China application no. 202410070565.0, filed on Jan. 17, 2024. The entirety of each of the above-mentioned patent applications is hereby incorporated by reference herein and made a part of this specification.

### TECHNICAL FIELD

The present application belongs to the technical field of polymer preparation, and relates to a preparation method of an aromatic hydrocarbon oligomer.

### RELATED ART

Mesophase pitch, which is a typical carbonaceous mesophase raw material, is an important precursor for obtaining pitch-based carbon fibers. Its quality directly determines the mechanical properties of the final carbon fiber product. Currently, raw materials for preparing mesophase pitch mainly include petroleum pitch, coal tar pitch, and pure aromatic hydrocarbon compounds. Among them, the pure aromatic hydrocarbon compounds are considered ideal raw materials for preparing high-quality mesophase pitch due to their simple molecular structure, containing only carbon and hydrogen elements, no ash content, and high aromaticity. When pure aromatic hydrocarbon compounds (e.g., naphthalene) are used to prepare mesophase pitch, they first need to be polymerized under the catalysis of an acidic catalyst to obtain a naphthalene oligomer, and the naphthalene oligomer is then subjected to high-temperature thermal polycondensation reaction to obtain mesophase pitch. Where the molecular weight and molecular weight distribution of the naphthalene oligomer both have a significant impact on the performance of the mesophase pitch. Generally, the more concentrated the molecular weight distribution of the naphthalene oligomer, the more concentrated the molecular weight distribution of the mesophase pitch, resulting in better homogeneity of the mesophase pitch and smaller viscosity fluctuation during the subsequent spinning process, enabling stable and continuous spinning operations to prepare a high-performance pitch-based carbon fiber. Therefore, how to prepare an aromatic hydrocarbon oligomer with controllable molecular weight and concentrated molecular weight distribution is of great significance for obtaining mesophase pitch with excellent spinning performance.

At present, there have been many reports on the preparation of mesophase pitch using a pure aromatic hydrocarbon compound as a raw material. For example, Patent CN102899061A discloses a method for preparing high-purity mesophase pitch, which uses refined naphthalene as the raw material and anhydrous aluminum trioxide as the catalyst, where the naphthalene is catalytically polymerized in an oil bath at 100-220° C. to obtain naphthalene pitch, which is then subjected to thermal polycondensation at 350-480° C. to obtain mesophase pitch. Patent CN1208065A discloses a method for preparing mesophase pitch using solid super acid, which uses a pure aromatic hydrocarbon or uses petroleum residual oil and petroleum pitch with a softening point of less than 200° C. as raw materials, and ZrO₂/SO₄²⁻ or TiO₂/SO₄²⁻ as the catalyst, to conduct an isothermal reaction at 90-300° C. to obtain an aromatic hydrocarbon oligomer, which is then pyrolyzed at 400-500° C. under normal pressure to prepare a corresponding mesophase pitch.

However, the above preparation methods lack control over the molecular weight and molecular weight distribution of aromatic hydrocarbon oligomers, and the prepared naphthalene oligomers have complex compositions. This leads to that the mesophase pitch prepared using them as raw materials has poor homogeneity, large viscosity fluctuation during the spinning process, difficulty in achieving stable and continuous spinning, and thus failure to obtain high-quality pitch-based carbon fiber products. Therefore, how to prepare an aromatic hydrocarbon oligomer with simpler and more regular composition and concentrated molecular weight distribution to provide a high-quality precursor for the preparation of high-quality mesophase pitch is an important issue faced by those skilled in the art.

### SUMMARY OF INVENTION

In view of the defects in the prior art, the present application provides a preparation method of an aromatic hydrocarbon oligomer. This method can efficiently prepare an aromatic hydrocarbon oligomer with simple composition and narrow molecular weight distribution under a mild condition, thereby providing a high-quality precursor for the preparation of high-quality mesophase pitch.

The present application provides a preparation method of an aromatic hydrocarbon oligomer, the preparation method including: subjecting an aromatic hydrocarbon compound to an oligomerization reaction under catalysis of a chloroaluminate ionic liquid to obtain the aromatic hydrocarbon oligomer;
where the oligomerization reaction is carried out in a solvent system, and the solvent is selected from chlorinated hydrocarbon solvents;
the aromatic hydrocarbon compound is selected from fused-ring aromatic hydrocarbons with 2 to 4 rings.

In the preparation method as described above, the chlorinated hydrocarbon solvent is selected from one or more of dichloromethane, trichloromethane, and dichloroethane.

In the preparation method as described above, the aromatic hydrocarbon compound is selected from one or more of naphthalene, methylnaphthalene, anthracene, phenanthrene, and pyrene.

In the preparation method as described above, the chloroaluminate ionic liquid has a molecular formula of Et₃NHCl-xAlCl₃, where 1<x≤2.

In the preparation method as described above, an amount of the chloroaluminate ionic liquid is 5 mol %-25 mol % of an amount of the aromatic hydrocarbon compound.

In the preparation method as described above, a temperature of the oligomerization reaction is 30-180° C.

In the preparation method as described above, time of the oligomerization reaction is 1-11 h.

In the preparation method as described above, the temperature of the oligomerization reaction is 30-90° C., and the time of the oligomerization reaction is 1-3 h.

In the preparation method as described above, a volume ratio of the solvent to the chloroaluminate ionic liquid is (50-500):1.

In the preparation method as described above, after completion of the oligomerization reaction, the method further includes a post-treatment process of the reaction system. The post-treatment includes: adding an alkali solution to the reaction solution after the completion of the oligomerization reaction to react with the ionic liquid in the reaction solution; then standing for layering to collect an organic phase; and subjecting the organic phase to water washing, concentrating, and drying treatments to obtain the aromatic hydrocarbon oligomer.

The implementation of the present application has at least the following beneficial effects:

In the preparation method of an aromatic hydrocarbon oligomer provided by the present application, an aromatic hydrocarbon compound is subjected to an oligomerization reaction in a chlorinated hydrocarbon solvent system under the catalysis of chloroaluminate ionic liquid, where the chlorinated hydrocarbon solvent has good solubility for both the chloroaluminate ionic liquid and the aromatic hydrocarbon compound; in addition, the chlorine atoms in the solvent form weak coordination with aluminum in the chloroaluminate ionic liquid, which reduces the acidity of the chloroaluminate ionic liquid, inhibits the occurrence of side reactions such as ring opening and cleavage, and improves the selectivity of the oligomerization reaction. Thus, the obtained naphthalene oligomer has the advantages of simple and regular composition and narrow molecular weight distribution. The mesophase pitch prepared using the naphthalene oligomer of the present application as raw material has high mesophase content and narrow molecular weight distribution, thereby having higher homogeneity and quality. This is conducive to obtaining high-quality carbon fiber products through continuous and stable spinning.

### BRIEF DESCRIPTION OF DRAWINGS

FIG. 1 is a comparative diagram of matrix-assisted laser desorption/ionization time-of-flight mass spectra of naphthalene oligomers of Example 1, Comparative Example 1, Comparative Example 2, and Comparative Example 3.

FIG. 2 is a comparative diagram of matrix-assisted laser desorption/ionization time-of-flight mass spectra of naphthalene oligomers catalyzed by different types of chloroaluminate ionic liquids.

FIG. 3 is a comparative diagram of matrix-assisted laser desorption/ionization time-of-flight mass spectra of naphthalene oligomers obtained in different amounts of catalyst.

FIG. 4 is a comparative diagram of matrix-assisted laser desorption/ionization time-of-flight mass spectra of naphthalene oligomers obtained at different polymerization temperatures.

FIG. 5 is a comparative diagram of matrix-assisted laser desorption/ionization time-of-flight mass spectra of naphthalene oligomers obtained at different polymerization times.

FIG. 6 is a matrix-assisted laser desorption/ionization time-of-flight mass spectrum of naphthalene oligomer obtained using trichloromethane as a solvent.

FIG. 7 is a matrix-assisted laser desorption/ionization time-of-flight mass spectrum of naphthalene oligomer obtained using dichloroethane as a solvent.

FIG. 8 is a matrix-assisted laser desorption/ionization time-of-flight mass spectrum of pyrene oligomer prepared in Example 9.

FIG. 9 is a matrix-assisted laser desorption/ionization time-of-flight mass spectrum of anthracene oligomer prepared in Example 10.

FIG. 10 is a time-of-flight mass spectrum of mesophase pitch prepared using the naphthalene oligomer of Example 1 as raw material.

FIG. 11 is a time-of-flight mass spectrum of mesophase pitch prepared using the naphthalene oligomer of Comparative Example 1 as raw material.

FIG. 12 is a time-of-flight mass spectrum of mesophase pitch prepared using the naphthalene oligomer of Comparative Example 2 as raw material.

FIG. 13 is a time-of-flight mass spectrum of mesophase pitch prepared using the naphthalene oligomer of Comparative Example 3 as raw material.

FIG. 14 is a polarized light micrograph of mesophase pitch prepared using the naphthalene oligomer of Example 1 as raw material.

FIG. 15 is a polarized light micrograph of mesophase pitch prepared using the naphthalene oligomer of Comparative Example 1 as raw material.

FIG. 16 is a polarized light micrograph of mesophase pitch prepared using the naphthalene oligomer of Comparative Example 2 as raw material.

FIG. 17 is a polarized light micrograph of mesophase pitch prepared using the naphthalene oligomer of Comparative Example 3 as raw material.

### DESCRIPTION OF EMBODIMENTS

To make the objectives, technical solutions, and advantages of the present application clearer, the technical solutions in the embodiments of the present application will be clearly and completely described below with reference to the examples of the present application. Obviously, the described examples are part of the embodiments of the present application, rather than all the embodiments. Based on the embodiments in the present application, all other embodiments obtained by those of ordinary skill in the art without creative efforts shall fall within the protection scope of the present application.

Pure aromatic hydrocarbon compounds have the characteristics of simple molecular structure, containing only carbon and hydrogen elements, no ash content, and high aromaticity, and thus are ideal raw materials for preparing high-quality mesophase pitch. The mesophase pitch can be prepared by subjecting the pure aromatic hydrocarbon compounds undergo oligomerization reaction to obtain oligomers followed by thermal polycondensation. Where, the molecular weight distribution of the oligomers has a significant impact on the processing performance of the mesophase pitch. Catalysts such as aluminum trichloride, hydrofluoric acid, and solid superacid are conventional catalysts for catalyzing the pure aromatic hydrocarbon compounds. However, these catalysts have defects such as low catalytic activity, high corrosiveness, and difficulty in separation from products. As a new type of catalyst, chloroaluminate ionic liquid has high reactivity of liquid acids and non-volatility of solid acids. Its structure and acidity are adjustable, and it exhibits high catalytic activity in the oligomerization reaction of the aromatic hydrocarbon compounds, significantly improving the preparation efficiency of the aromatic hydrocarbon oligomers. Furthermore, compared with the aforementioned conventional catalysts, it has the advantages of environmental friendliness, low corrosiveness, and reusability. However, although the chloroaluminate ionic liquid can significantly improve the preparation efficiency of the aromatic hydrocarbon oligomers, the obtained aromatic hydrocarbon oligomers have disadvantages such as complex composition, many by-products from ring opening and cleavage, and wide molecular weight distribution, which are not conducive to obtaining high-quality mesophase pitch.

Based on this, the present application provides a preparation method of an aromatic hydrocarbon oligomer, including: subjecting an aromatic hydrocarbon compound to an oligomerization reaction under catalysis of a chloroaluminate ionic liquid to obtain an aromatic hydrocarbon oligomer;
wherein the oligomerization reaction is carried out in a solvent system, and the solvent is selected from chlorinated hydrocarbon solvents; and the aromatic hydrocarbon compound is selected from fused-ring aromatic hydrocarbons with 2 to 4 rings.

The inventors found that when the above oligomerization reaction uses a chlorinated hydrocarbon solvent as the reaction solvent, aromatic hydrocarbon oligomers with narrow molecular weight distribution, simple composition, and few by-products can be obtained with high yield, which is conducive to the preparation of high-quality mesophase pitch. The reason may be as follows: on one hand, the chlorine atoms in the chlorinated hydrocarbon solvent form weak coordination with aluminum in the chloroaluminate ionic liquid, reducing its acidity, inhibiting the occurrence of side reactions such as ring opening reaction, cleavage reaction, and hydrogen transfer reaction, thereby improving the selectivity of the oligomerization reaction and thus facilitating the acquisition of aromatic hydrocarbon oligomers with narrow molecular weight distribution and simple and regular composition; on the other hand, the chlorinated hydrocarbon solvent has good solubility for both the chloroaluminate ionic liquid and the aromatic hydrocarbon compound, enabling the reaction system to proceed under homogeneous conditions. The homogeneous conditions can not only promote the efficient progress of the reaction but also adjust the viscosity of the reaction system and the concentrations of the catalyst and substrate, thereby helping to improve the selectivity of the oligomerization reaction.

During the research process, the inventors also tried common organic solvents such as toluene, xylene, trimethylbenzene, dimethyl sulfoxide (DMSO), N,N-dimethylformamide (DMF), ethanol, n-hexane, petroleum ether, isobutane, and cyclohexane. Among them, solvents such as DMSO, DMF, and ethanol will react with the chloroaluminate ionic liquid, making it difficult for the chloroaluminate ionic liquid to exert its catalytic effect; alkane solvents such as n-hexane, petroleum ether, isobutane, and cyclohexane cannot play a dissolving role because they have poor solubility for both the chloroaluminate ionic liquid and the oligomers; solvents such as toluene, xylene, and trimethylbenzene have poor solubility for the chloroaluminate ionic liquid, making it difficult for the reaction to proceed in a homogeneous system, and as a result, the preparation efficiency of aromatic hydrocarbon oligomers is low, and the prepared aromatic hydrocarbon oligomers have complex composition and wide molecular weight distribution.

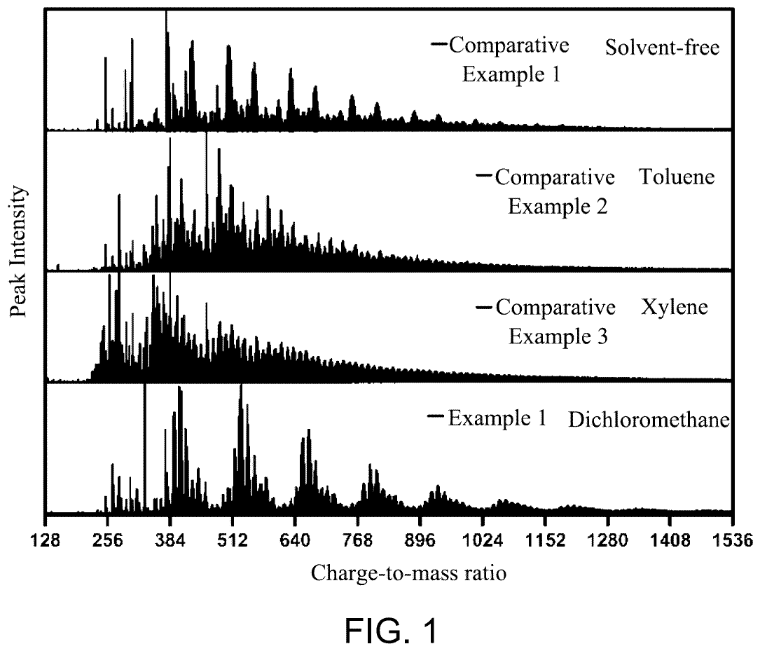

The composition information and molecular weight distribution of the above aromatic hydrocarbon oligomers can be analyzed using a matrix-assisted laser desorption/ionization time-of-flight mass spectrometer. FIG. 1 shows matrix-assisted laser desorption/ionization time-of-flight mass spectra. Taking FIG. 1 as an example, the composition information and molecular weight distribution of naphthalene oligomers are analyzed as follows: 10 peak clusters can be seen in the mass spectra of Example 1, representing the 2- to 9-mers of naphthalene respectively. Among them, the peak cluster with m/z in the range of 254-350 is the naphthalene dimer, the peak cluster with m/z in the range of 350-490 is the naphthalene trimer, the peak cluster with m/z in the range of 490-620 is the naphthalene tetramer, the peak cluster with m/z in the range of 620-740 is the naphthalene pentamer, the peak cluster with m/z in the range of 740-870 is the naphthalene hexamer, the peak cluster with m/z in the range of 870-1020 is the naphthalene heptamer, and the three peak clusters with m/z in the range of 1020-1400 are the naphthalene octamer, nonamer, and decamer respectively from left to right. It can be seen from FIG. 1 that the peak intensity decreases sequentially from left to right, and especially, the peak intensity of the 8- to 10-mers of naphthalene is obviously very weak, indicating that their content in the naphthalene oligomers is very low. Therefore, the composition of the naphthalene oligomer in Example 1 mainly includes 2- to 7-mers. The molecular weight range of the naphthalene oligomers in Comparative Examples 1-3 is basically the same as that in Example 1, but the boundaries between the peak clusters in the mass spectra of Example 1 are clear, while the molecular weight distribution of Comparative Examples 1-3 is scattered, and the peak clusters of naphthalene multi-mers cannot be clearly seen. This indicates that the naphthalene oligomers of Comparative Examples 1-3 have more complex composition compared with Example 1, and may contain many undesired substances generated through side reactions such as ring opening and cleavage.

The present application has no special limitation on the chlorinated hydrocarbon solvent, which can be selected from low-carbon chlorinated hydrocarbon solvents commonly used in the art, including but not limited to one or more of dichloromethane, trichloromethane, and dichloroethane.

The inventors found through research that the amount of solvent has no obvious impact on the yield and selectivity of the reaction. Therefore, the present application has no special limitation on the amount of solvent. In an embodiment, a volume ratio of the solvent to the chloroaluminate ionic liquid is (50-500):1. When the amount of solvent meets the above ratio range, it not only enables the reaction to proceed under homogeneous conditions, but also ensures the chloroaluminate ionic liquid has an appropriate catalytic concentration, further ensuring that the oligomerization reaction has high catalytic activity and reaction selectivity.

The aromatic hydrocarbon compound of the present application is a fused-ring aromatic hydrocarbon with 2 to 4 rings, including but not limited to one or more of naphthalene, methylnaphthalene, anthracene, phenanthrene, and pyrene. In an embodiment, the fused-ring aromatic hydrocarbon is naphthalene. The oligomers prepared using the above aromatic hydrocarbon compounds all have the advantages of simple and regular composition and narrow molecular weight distribution, and are high-quality raw materials for preparing high-quality mesophase pitch.

Chloroaluminate ionic liquid is a Lewis acid catalyst, whose acidity originates from [AlₓCl₃ₓ₊₁]⁻ (e.g., [AlCl₄]⁻ and [Al₂Cl₇]⁻), and its cation can derive from ammonium salts.

In a specific embodiment, the chloroaluminate ionic liquid used in the present application has a molecular formula of Et₃NHCl-xAICI₃, where 1<x≤2. Here, x can be selected from 1.1, 1.2, 1.4, 1.6, 1.8, 2.0, or a range composed of any two of the above values. Chloroaluminate ionic liquids with the above composition can all efficiently catalyze the oligomerization reaction of aromatic hydrocarbon compounds, and the obtained aromatic hydrocarbon oligomers all have the advantages of simple and regular composition and narrow molecular weight distribution.

The chloroaluminate ionic liquid with the above composition can be obtained by the reaction of anhydrous aluminum chloride and triethylammonium chloride. Specifically, the value of x can be controlled by controlling the molar ratio of anhydrous aluminum chloride to triethylammonium chloride in the reaction. For example, the molar ratio of anhydrous aluminum chloride to triethylammonium chloride in the reaction can be controlled to 1:1.2 to prepare an ionic liquid with a molecular formula of Et₃NHCl-1.2AlCl₃.

In a specific embodiment, the amount of the chloroaluminate ionic liquid is 5 mol %-25 mol % of the amount of the aromatic hydrocarbon compound, specifically 5 mol %, 10 mol %, 15 mol %, 20 mol %, 25 mol %, or a range composed of any two of the above values.

The highly conjugated structure of fused-ring aromatic hydrocarbons makes them highly stable, and the preparation of oligomers through polycondensation can only be achieved at relatively high temperatures. The introduction of a catalyst can reduce the energy required for the oligomerization reaction, thereby reducing the harshness of the reaction and energy consumption, and enabling the reaction to proceed at a lower temperature. However, in the previously reported reactions for catalyzing the oligomerization of aromatic hydrocarbon compounds using chloroaluminate ionic liquid, the reaction temperature is generally not lower than 100° C. However, by introducing a chlorinated hydrocarbon solvent into the reaction system, the present application enables the reaction to proceed efficiently at a temperature of lower than 100° C. Even at 30° C., aromatic hydrocarbon oligomers with simple composition and narrow molecular weight distribution can be efficiently obtained with high yield. This greatly reduces the harshness of the reaction and energy consumption. Specifically, the oligomerization reaction of the present application can proceed smoothly within a temperature range of 30-180° C.

In a specific embodiment, the time of the oligomerization reaction of the present application is 1-11 h, for example, 1 h, 3 h, 4 h, 5 h, 6 h, 7 h, 9 h, 11 h, or a range composed of any two of the above values.

Further, the oligomerization reaction of the present application can prepare aromatic hydrocarbon oligomers with high yield and high selectivity under the conditions of a temperature of 30-90° C. and a time of 1-3 h. The reaction under the above conditions can further reduce the energy consumption of the reaction and improve the preparation efficiency.

The present application has no special limitation on the pressure of the oligomerization reaction, which can be carried out under normal pressure, and the above reaction process can be completed using a reaction kettle as the container. To avoid the interference of other impurities as much as possible, the oligomerization reaction can be carried out under the protection of an inert gas.

After the completion of the oligomerization reaction, the method further includes a post-treatment process for the reaction system. In a specific embodiment, the post-treatment includes: adding an alkali solution to the reaction solution after the completion of the oligomerization reaction, to react with the ionic liquid in the reaction solution; then standing for layering, to collect the organic phase; and subjecting the organic phase to water washing, concentrating, and drying treatments to obtain the aromatic hydrocarbon oligomer.

Further, the alkali solution can be selected from a sodium hydroxide solution or a potassium hydroxide solution, with a concentration of 0.5-2 mol/L.

After being added to the reaction solution, the alkali solution will react with AlCl₃ in the ionic liquid structure, first converting it into a solid aluminum hydroxide precipitate, and then continuing reaction to convert it into meta-aluminate dissolved in the inorganic phase. After standing for layering, the organic phase containing aromatic hydrocarbon oligomers is collected, and then the organic phase is washed with water until it is neutral. The reaction solvent is removed by concentration, and the residual water is removed by drying, to obtain the aromatic hydrocarbon oligomer.

In summary, the preparation method of an aromatic hydrocarbon oligomer provided by the present application, by causing an aromatic hydrocarbon compound to undergo oligomerization reaction in a chlorinated hydrocarbon solvent system under the catalysis of chloroaluminate ionic liquid, can obtain an aromatic hydrocarbon oligomer with narrow molecular weight distribution and simple and regular composition with high yield. The aromatic hydrocarbon oligomer can be used to prepare mesophase pitch through a simple thermal polycondensation reaction. The prepared mesophase pitch has the advantages of high mesophase content, narrow molecular weight distribution, and good homogeneity, which is conducive to stable and continuous spinning operations, and thus the preparation of carbon fiber products with excellent performance.

The following will introduce the preparation method of an aromatic hydrocarbon oligomer provided by the present application in detail with reference to specific examples.

In the following examples, unless otherwise specified, all raw materials can be obtained through commercial purchase or conventional preparation methods; for experimental methods without specified specific conditions, specific selections are made according to conventional methods and conditions in the field or according to the product instructions.

### Example 1

This example provides a preparation method of a naphthalene oligomer, including the following steps:
1) Under normal pressure and nitrogen protection, 10 g of naphthalene (1 eq.) and 1.58 g (1 mL) of Et₃NHCl-2.0AlCl₃ (25 mol %) were added into a high-pressure reaction kettle lined with polytetrafluoroethylene (PTFE), and 50 mL of dichloromethane as reaction solvent was added to carry out catalytic polymerization reaction at 160° C.
2) After the reaction was completed, the reaction mixture was transferred to a glass beaker, and a 2 mol/L aqueous NaOH solution was slowly dropped into it, with solid precipitation occurring. During this period, a glass rod was used for continuous stirring. After the solid precipitation completely disappeared, the addition of the aqueous NaOH solution was stopped. After standing for layering, the organic phase was collected. Deionized water was used to wash the organic phase until it was neutral. Finally, dichloromethane solvent and water in the organic phase were removed by rotary evaporation and vacuum drying to obtain a naphthalene oligomer with a yield of 48%. Samples were taken to analyze the composition of the naphthalene oligomer using a matrix-assisted laser desorption/ionization time-of-flight mass spectrometer. FIG. 1 is a comparative diagram of matrix-assisted laser desorption/ionization time-of-flight mass spectra of naphthalene oligomers of Example 1, Comparative Example 1, Comparative Example 2, and Comparative Example 3. It can be seen from FIG. 1 that the composition of the naphthalene oligomer in this example mainly includes 2- to 7-mers of naphthalene, with clear boundaries between the multi-mer peak clusters.

It should be noted that the composition of the naphthalene oligomers listed in this example and the following examples refers to their main composition, not the absolute composition. Taking this example as an example, the composition of the naphthalene oligomer mainly includes 2- to 7-mers of naphthalene, but it does not exclude that it also contains oligomers with higher polymerization degrees in low content.

### Example 2: Screening of Type of Catalyst

The preparation of naphthalene oligomers of this example was carried out with reference to the preparation method of Example 1, and the type of catalyst was screened. The specific reaction conditions are listed in Table 1.

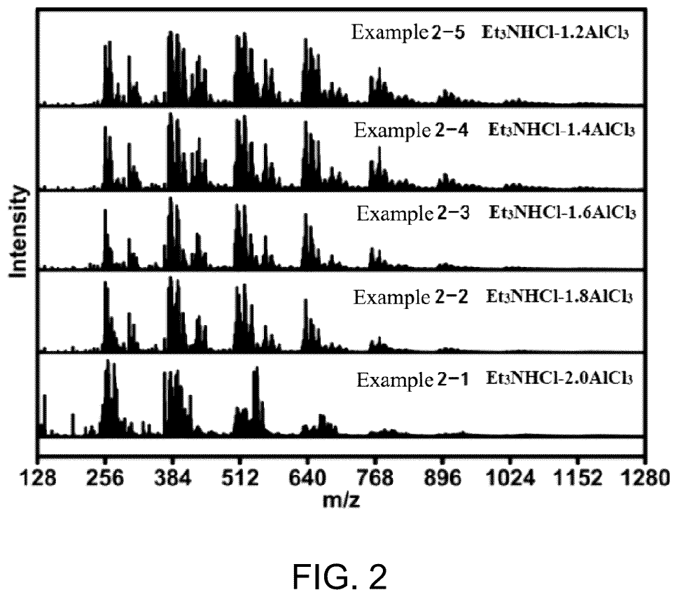

The yield of naphthalene oligomers was calculated, and samples were taken to analyze the composition of the naphthalene oligomers using a matrix-assisted laser desorption/ionization time-of-flight mass spectrometer. FIG. 2 is a comparative diagram of matrix-assisted laser desorption/ionization time-of-flight mass spectra of naphthalene oligomers catalyzed by different types of chloroaluminate ionic liquids. It can be seen from FIG. 2 that the naphthalene oligomers catalyzed by chloroaluminate ionic liquids with x in the range of 1.2-2.0 have simple and regular composition, with clear boundaries between the multi-mer peak clusters. The yield and composition of the naphthalene oligomers are listed in Table 1.

TABLE 1

| | | | | Amount of | | Volume | | Composition |
|---|---|---|---|---|---|---|---|---|
| | | Polymerization | Polymerization | Catalyst/mol | Solvent | Ratio of | Yield of | of |
| | | Temperature of | Time of | % (based on | for | Solvent to | Naphthalene | Naphthalene |
| | Type of Catalyst | Naphthalene/° C. | Naphthalene/h | naphthalene) | Reaction | Catalyst | Oligomer/% | Oligomer |
| Example 2-1 | Et₃NHCl—1.2AlCl₃ | 30 | 1 | 5 | CH₂Cl₂ | 50:1 | 3 | 2- to 4-mers of |
| | | | | | | | | naphthalene |
| Example 2-2 | Et₃NHCl—1.4AlCl₃ | 30 | 1 | 5 | CH₂Cl₂ | 50:1 | 10 | 2- to 5-mers of |
| | | | | | | | | naphthalene |
| Example 2-3 | Et₃NHCl—1.6AlCl₃ | 30 | 1 | 5 | CH₂Cl₂ | 50:1 | 11 | 2- to 5-mers of |
| | | | | | | | | naphthalene |
| Example 2-4 | Et₃NHCl—1.8AlCl₃ | 30 | 1 | 5 | CH₂Cl₂ | 50:1 | 15 | 2- to 6-mers of |
| | | | | | | | | naphthalene |
| Example 2-5 | Et₃NHCl—2.0AlCl₃ | 30 | 1 | 5 | CH₂Cl₂ | 50:1 | 19 | 2- to 6-mers of |
| | | | | | | | | naphthalene |

It can be seen from Table 1 that as x increases, the yield of naphthalene oligomers also increases.

### Example 3: Screening of Amount of Catalyst

The preparation of naphthalene oligomers of this example was carried out with reference to the preparation method of Example 1, and the amount of catalyst was screened. The specific reaction conditions are listed in Table 2.

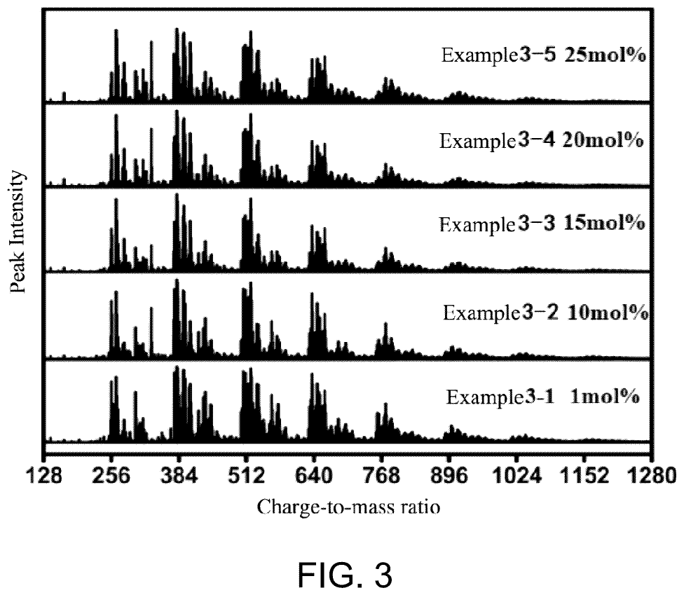

The yield of naphthalene oligomers was calculated, and samples were taken to analyze the composition of the naphthalene oligomers using a matrix-assisted laser desorption/ionization time-of-flight mass spectrometer. FIG. 3 is a comparative diagram of matrix-assisted laser desorption/ionization time-of-flight mass spectra of naphthalene oligomers obtained under different amounts of catalyst. It can be seen from FIG. 3 that the naphthalene oligomers obtained when the amount of catalyst is in the range of 1 mol %-25 mol % have simple and regular composition, with clear boundaries between the multi-mer peak clusters. The yield and composition of the naphthalene oligomers are listed in Table 2.

TABLE 2

| | | | | Amount of | | | | Composition |
|---|---|---|---|---|---|---|---|---|
| | | Polymerization | Polymerization | Catalyst/mol | Solvent | | Yield of | of |
| | | Temperature of | Time of | % (based on | for | Amount of | Naphthalene | Naphthalene |
| | Type of Catalyst | Naphthalene/° C. | Naphthalene/h | naphthalene) | Reaction | Solvent/mL | Oligomer/% | Oligomer |
| Example 3-1 | Et₃NHCl—2.0AlCl₃ | 30 | 1 | 1 | CH₂Cl₂ | 50:1 | 5 | 2- to 5-mers of |
| | | | | | | | | naphthalene |
| Example 3-2 | Et₃NHCl—2.0AlCl₃ | 30 | 1 | 10 | CH₂Cl₂ | 50:1 | 26 | 2- to 5-mers of |
| | | | | | | | | naphthalene |
| Example 3-3 | Et₃NHCl—2.0AlCl₃ | 30 | 1 | 15 | CH₂Cl₂ | 50:1 | 39 | 2- to 5-mers of |
| | | | | | | | | naphthalene |
| Example 3-4 | Et₃NHCl—2.0AlCl₃ | 30 | 1 | 20 | CH₂Cl₂ | 50:1 | 47 | 2- to 5-mers of |
| | | | | | | | | naphthalene |
| Example 3-5 | Et₃NHCl—2.0AlCl₃ | 30 | 1 | 25 | CH₂Cl₂ | 50:1 | 56 | 2- to 5-mers of |
| | | | | | | | | naphthalene |

It can be seen from Table 2 that as the amount of catalyst increases, the yield of naphthalene oligomers gradually increases.

### Example 4: Screening of Polymerization Temperature

The preparation of naphthalene oligomers of this example was carried out with reference to the preparation method of Example 1, and the polymerization temperature was screened. The specific reaction conditions are listed in Table 3.

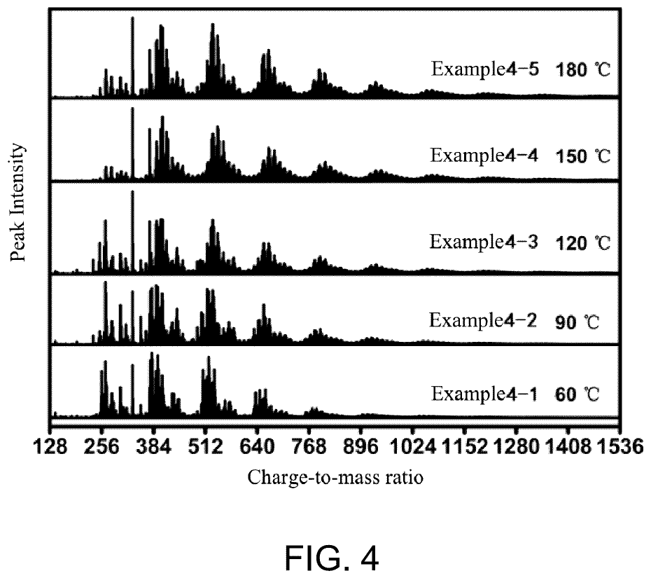

The yield of naphthalene oligomers was calculated, and samples were taken to analyze the composition of the naphthalene oligomers using a matrix-assisted laser desorption/ionization time-of-flight mass spectrometer. FIG. 4 is a comparative diagram of matrix-assisted laser desorption/ionization time-of-flight mass spectra of naphthalene oligomers obtained at different polymerization temperatures. Based on FIG. 4 and combined with FIG. 2 and FIG. 3, it can be seen that the naphthalene oligomers obtained within the temperature range of 30-180° C. have simple and regular composition, with clear boundaries between the multi-mer peak clusters. As the reaction temperature increases, the polymerization degree of naphthalene also increases, and the content of naphthalene hexamers and heptamers increases, while the content of 2- to 5-mers of naphthalene does not decrease significantly. This indicates that as the temperature increases, the molecular weight distribution of naphthalene oligomers will become wider. The yield and composition of the naphthalene oligomers are listed in Table 3.

TABLE 3

| | | | | Amount of | | | | Composition |
|---|---|---|---|---|---|---|---|---|
| | | Polymerization | Polymerization | Catalyst/mol | Solvent | | Yield of | of |
| | | Temperature of | Time of | % (based on | for | Amount of | Naphthalene | Naphthalene |
| | Type of Catalyst | Naphthalene/° C. | Naphthalene/h | naphthalene) | Reaction | Solvent/mL | Oligomer/% | Oligomer |
| Example 4-1 | Et₃NHCl—2.0AlCl₃ | 60 | 1 | 5 | CH₂Cl₂ | 50:1 | 20 | 2- to 6-mers of |
| | | | | | | | | naphthalene |
| Example 4-2 | Et₃NHCl—2.0AlCl₃ | 90 | 1 | 5 | CH₂Cl₂ | 50:1 | 25 | 2- to 6-mers of |
| | | | | | | | | naphthalene |
| Example 4-3 | Et₃NHCl—2.0AlCl₃ | 120 | 1 | 5 | CH₂Cl₂ | 50:1 | 33 | 2- to 7-mers of |
| | | | | | | | | naphthalene |
| Example 4-4 | Et₃NHCl—2.0AlCl₃ | 150 | 1 | 5 | CH₂Cl₂ | 50:1 | 45 | 3- to 6-mers of |
| | | | | | | | | naphthalene |
| Example 4-5 | Et₃NHCl—2.0AlCl₃ | 180 | 1 | 5 | CH₂Cl₂ | 50:1 | 55 | 3- to 7-mers of |
| | | | | | | | | naphthalene |

It can be seen from Table 3 that as the reaction temperature increases, the yield of naphthalene oligomers also increases.

### Example 5: Screening of Polymerization Time

The preparation of naphthalene oligomers of this example was carried out with reference to the preparation method of Example 1, and the polymerization time was screened. The specific reaction conditions are listed in Table 4.

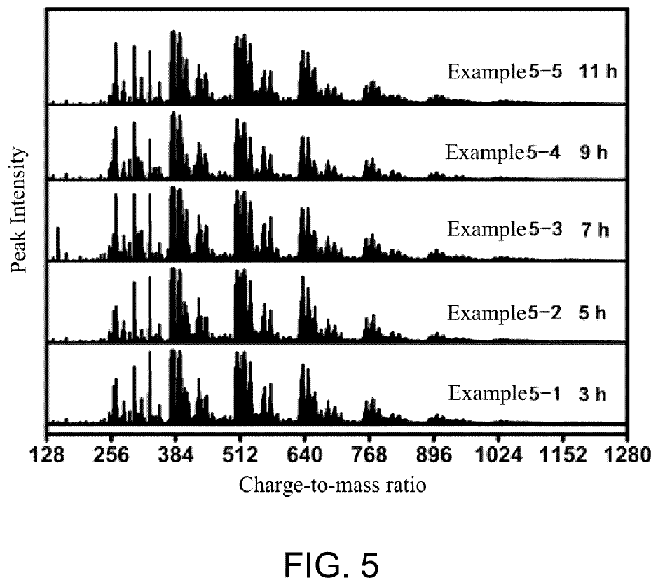

The yield of naphthalene oligomers was calculated, and samples were taken to analyze the composition of the naphthalene oligomers using a matrix-assisted laser desorption/ionization time-of-flight mass spectrometer. FIG. 5 is a comparative diagram of matrix-assisted laser desorption/ionization time-of-flight mass spectra of naphthalene oligomers obtained under different polymerization times. It can be seen from FIG. 5 that within the reaction time range of 1-11 h, naphthalene oligomers with a polymerization degree of 2-5, clear boundaries between multi-mer peak clusters, and simple and regular composition can be obtained. The yield and composition of the naphthalene oligomers are listed in Table 4.

TABLE 4

| | | | | Amount of | | Volume | | Composition |
|---|---|---|---|---|---|---|---|---|
| | | Polymerization | Polymerization | Catalyst/mol | Solvent | Ratio of | Yield of | of |
| | | Temperature of | Time of | % (based on | for | Solvent to | Naphthalene | Naphthalene |
| | Type of Catalyst | Naphthalene/° C. | Naphthalene/h | naphthalene) | Reaction | Catalyst | Oligomer/% | Oligomer |
| Example 5-1 | Et₃NHCl—2.0AlCl₃ | 30 | 3 | 5 | CH₂Cl₂ | 50:1 | 15 | 2- to 5-mers of |
| | | | | | | | | naphthalene |
| Example 5-2 | Et₃NHCl—2.0AlCl₃ | 30 | 5 | 5 | CH₂Cl₂ | 50:1 | 17 | 2- to 5-mers of |
| | | | | | | | | naphthalene |
| Example 5-3 | Et₃NHCl—2.0AlCl₃ | 30 | 7 | 5 | CH₂Cl₂ | 50:1 | 19 | 2- to 5-mers of |
| | | | | | | | | naphthalene |
| Example 5-4 | Et₃NHCl—2.0AlCl₃ | 30 | 9 | 5 | CH₂Cl₂ | 50:1 | 21 | 2- to 5-mers of |
| | | | | | | | | naphthalene |
| Example 5-5 | Et₃NHCl—2.0AlCl₃ | 30 | 11 | 5 | CH₂Cl₂ | 50:1 | 24 | 2- to 5-mers of |
| | | | | | | | | naphthalene |

It can be seen from Table 4 that as the polymerization time increases, the yield of naphthalene oligomers also increases.

### Example 6: Screening of Amount of Solvent for Reaction

The preparation of naphthalene oligomers of this example was carried out with reference to the preparation method of Example 1, and amount of solvent for the reaction was screened. The specific reaction conditions and the yield of the naphthalene oligomers are listed in Table 5.

TABLE 5

| | | | | Amount for | | Volume | |
|---|---|---|---|---|---|---|---|
| | | Polymerization | Polymerization | Catalyst/mol | Solvent | Ratio of | Yield of |
| | | Temperature of | Time of | % (based on | for | Solvent to | Naphthalene |
| | Type of Catalyst | Naphthalene/° C. | Naphthalene/h | naphthalene) | Reaction | Catalyst | Oligomer/% |
| Example 6-1 | Et₃NHCl—2.0AlCl₃ | 30 | 1 | 5 | CH₂Cl₂ | 50:1 | 19 |
| Example 6-2 | Et₃NHCl—2.0AlCl₃ | 30 | 1 | 5 | CH₂Cl₂ | 100:1 | 19 |
| Example 6-3 | Et₃NHCl—2.0AlCl₃ | 30 | 1 | 5 | CH₂Cl₂ | 250:1 | 19 |
| Example 6-4 | Et₃NHCl—2.0AlCl₃ | 30 | 1 | 5 | CH₂Cl₂ | 400:1 | 19 |
| Example 6-5 | Et₃NHCl—2.0AlCl₃ | 30 | 1 | 5 | CH₂Cl₂ | 500:1 | 18 |

It can be seen from Table 5 that the amount of solvent has no obvious impact on the reaction yield.

### Example 7

This example provides a preparation method of a naphthalene oligomer, which is basically the same as the preparation steps of Example 1, except that the reaction solvent is replaced with trichloromethane. The catalytic polymerization of naphthalene is carried out under the same reaction conditions to obtain a naphthalene oligomer.

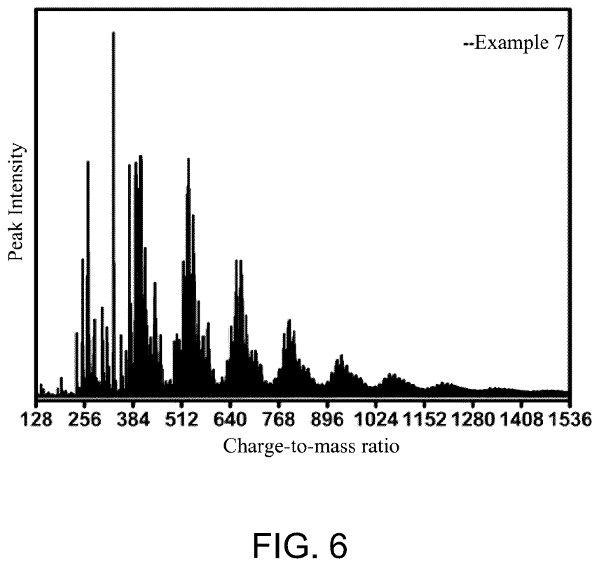

The yield of the naphthalene oligomer in this example is 47%. A sample of the naphthalene oligomer in this example was taken and analyzed using a matrix-assisted laser desorption/ionization time-of-flight mass spectrometer. FIG. 6 is a matrix-assisted laser desorption/ionization time-of-flight mass spectrum of the naphthalene oligomer prepared in Example 7. It can be seen from FIG. 6 that the multi-mers of the naphthalene oligomer have clear boundaries therebetween, and mainly are 2- to 6-mers of naphthalene.

### Example 8

This example provides a preparation method of a naphthalene oligomer, which is basically the same as the preparation steps of Example 1, except that the solvent for reaction is replaced with dichloroethane. The catalytic polymerization of naphthalene is carried out under the same reaction conditions to obtain a naphthalene oligomer.

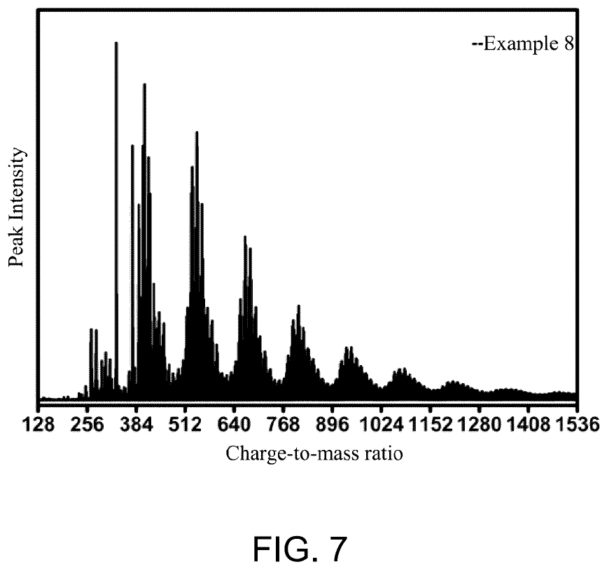

The yield of the naphthalene oligomer in this example is 46%. A sample of the naphthalene oligomer in this example was taken and analyzed using a matrix-assisted laser desorption/ionization time-of-flight mass spectrometer. FIG. 7 is a matrix-assisted laser desorption/ionization time-of-flight mass spectrum of the naphthalene oligomer prepared in Example 8. It can be seen from FIG. 7 that the multi-mers of the naphthalene oligomer have clear boundaries therebetween, and mainly are 2- to 6-mers of naphthalene.

### Example 9

This example provides a preparation method of a pyrene oligomer, which is basically the same as the preparation steps of Example 1, except that naphthalene as polymerization raw material is replaced with pyrene. The catalytic polymerization of pyrene is carried out under the same reaction conditions to obtain a pyrene oligomer.

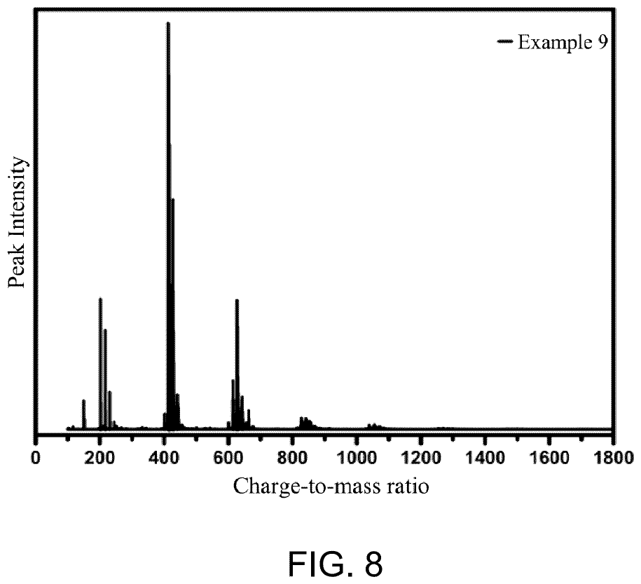

The yield of the pyrene oligomer in this example is 70%. A sample of the pyrene oligomer in this example was taken and analyzed using a matrix-assisted laser desorption/ionization time-of-flight mass spectrometer. FIG. 8 is a matrix-assisted laser desorption/ionization time-of-flight mass spectrum of the pyrene oligomer prepared in Example 9. It can be seen from FIG. 8 that the pyrene oligomer is 2- to 4-mers of pyrene, with clear boundaries between the multi-mers.

### Example 10

This example provides a preparation method of an anthracene oligomer, which is basically the same as the preparation steps of Example 1, except that the naphthalene as polymerization raw material is replaced with anthracene. The catalytic polymerization of anthracene is carried out under the same reaction conditions to obtain an anthracene oligomer.

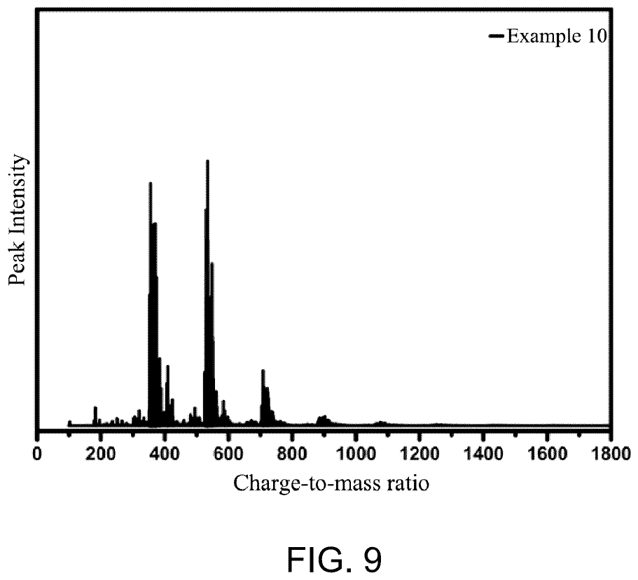

The yield of anthracene oligomer in this example is 57%. A sample of the anthracene oligomer in this example was taken and analyzed using a matrix-assisted laser desorption/ionization time-of-flight mass spectrometer. FIG. 9 is a matrix-assisted laser desorption/ionization time-of-flight mass spectrum of the anthracene oligomer prepared in Example 10. It can be seen from FIG. 9 that the anthracene oligomer is 2- to 4-mers of anthracene, with clear boundaries between the multi-mers.

### Comparative Example 1

This comparative example provides a preparation method of a naphthalene oligomer, which is basically the same as the preparation steps of Example 1, except that the preparation of naphthalene oligomer in this comparative example is carried out without a solvent, and the other conditions are the same as those in Example 1. The yield of the naphthalene oligomer in this comparative example is 36%.

### Comparative Example 2

This comparative example provides a preparation method of a naphthalene oligomer, which is basically the same as the preparation steps of Example 1, except that dichloromethane as reaction solvent is replaced with toluene. The catalytic polymerization of naphthalene is carried out under the same reaction conditions to obtain a naphthalene oligomer. The yield of the naphthalene oligomer in this comparative example is 35%.

### Comparative Example 3

This comparative example provides a preparation method of a naphthalene oligomer, which is basically the same as the preparation steps of Example 1, except that dichloromethane as the reaction solvent is replaced with xylene. The catalytic polymerization of naphthalene is carried out under the same reaction conditions to obtain a naphthalene oligomer. The yield of the naphthalene oligomer in this comparative example is 38%.

Samples of the naphthalene oligomers from Comparative Examples 1, 2, and 3 were respectively taken and analyzed using a matrix-assisted laser desorption/ionization time-of-flight mass spectrometer. FIG. 1 is a comparative diagram of matrix-assisted laser desorption/ionization time-of-flight mass spectra of the naphthalene oligomers of Example 1, Comparative Example 1, Comparative Example 2, and Comparative Example 3. It can be seen from FIG. 1 that compared with using dichloromethane as the solvent, the naphthalene oligomers obtained when using no solvent or using toluene or xylene as the solvent have relatively scattered molecular weight distribution, and the peak clusters of multi-mers of naphthalene are not clear, indicating that the composition of the naphthalene oligomers is complex.

### Test Example

Mesophase pitch was prepared using the naphthalene oligomers prepared in Example 1, Comparative Example 1, Comparative Example 2, and Comparative Example 3 as raw materials respectively. The preparation method was as follows: 120 g of the naphthalene oligomers was added into a reaction kettle, the air in the kettle was purged with nitrogen, then the reaction kettle was sealed, and thermal polycondensation reaction was carried out at 400° C. for 4 h to prepare mesophase pitch. The preparation yield is shown in Table 6.

TABLE 6

| | | Comparative | Comparative | Comparative |
|---|---|---|---|---|
| | Example 1 | Example 1 | Example 2 | Example 3 |
| Yield/% | 68 | 53 | 52 | 50 |

It can be seen from Table 6 that the yield of mesophase pitch obtained in Example 1 is significantly higher than that in Comparative Examples 1-3.

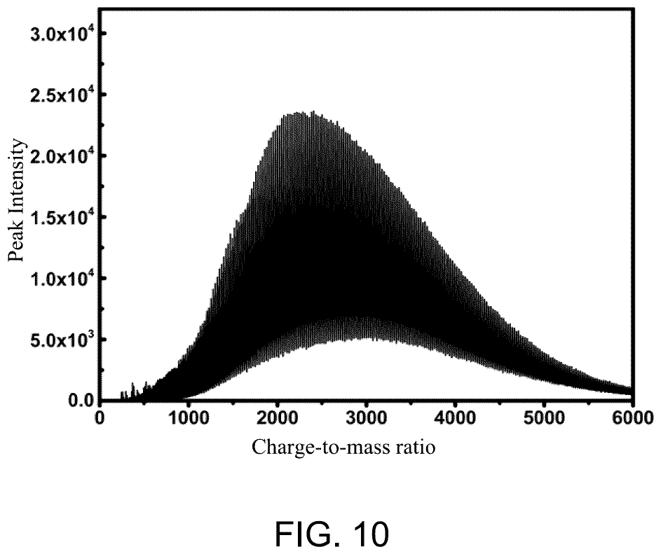

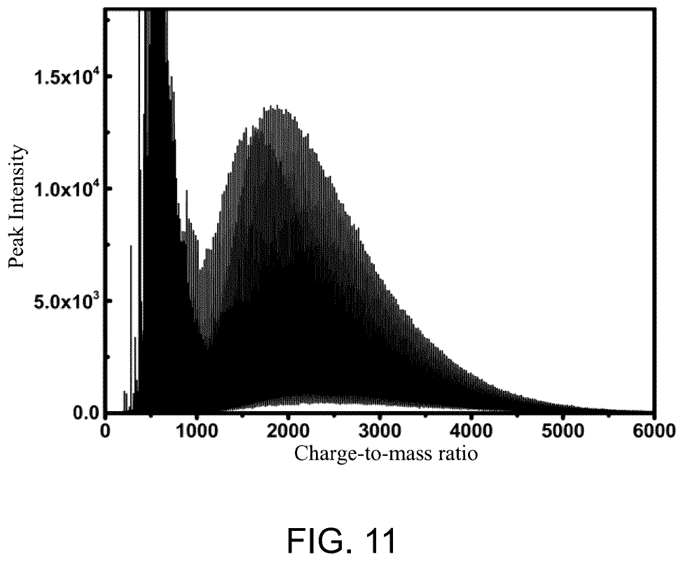

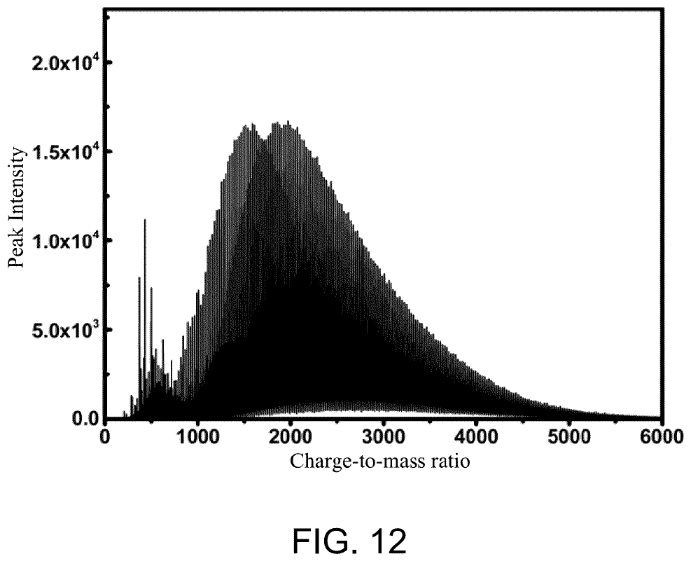

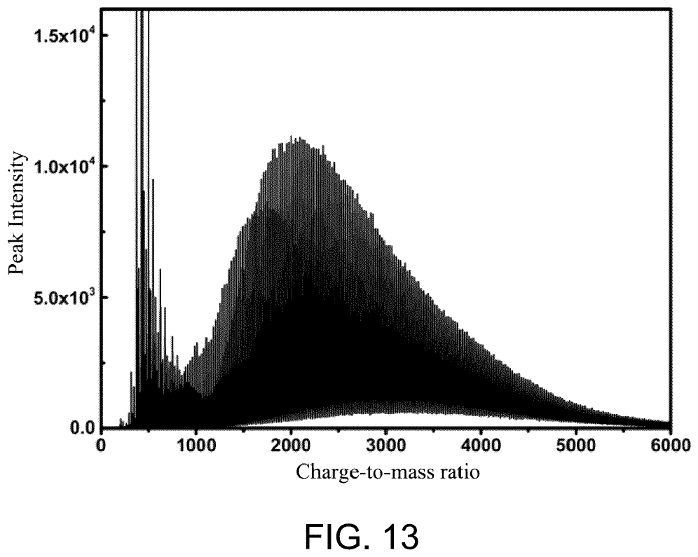

Samples of the prepared mesophase pitch were taken and analyzed for their molecular weight and molecular weight distribution using a time-of-flight mass spectrometer respectively. FIG. 10 is a time-of-flight mass spectrum of mesophase pitch prepared using the naphthalene oligomer of Example 1 as raw material, FIG. 11 is a time-of-flight mass spectrum of mesophase pitch prepared using the naphthalene oligomer of Comparative Example 1 as raw material, FIG. 12 is a time-of-flight mass spectrum of mesophase pitch prepared using the naphthalene oligomer of Comparative Example 2 as raw material, and FIG. 13 is a time-of-flight mass spectrum of mesophase pitch prepared using the naphthalene oligomer of Comparative Example 3 as raw material. It can be seen from FIG. 10-FIG. 13 that the molecular weight of the mesophase pitch prepared using the naphthalene oligomer of Example 1 as raw material is distributed in the range of 500-6000, mainly concentrated in the range of 1500-4000; the molecular weight of the mesophase pitch prepared using the naphthalene oligomers of Comparative Examples 1-3 as raw materials is distributed in the range of 300-6000, mainly concentrated in the range of 400-4000. It can be seen from this that the molecular weight distribution of Example 1 is obviously narrower than that of Comparative Examples 1-3, and the mesophase pitch with a more concentrated molecular weight distribution also has better spinning stability, showing the advantage of the mesophase pitch of Example 1.

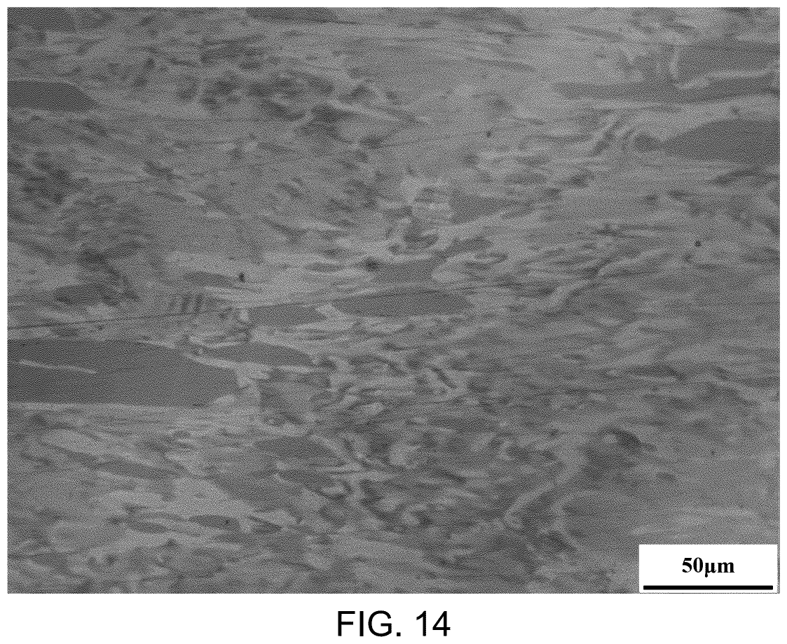

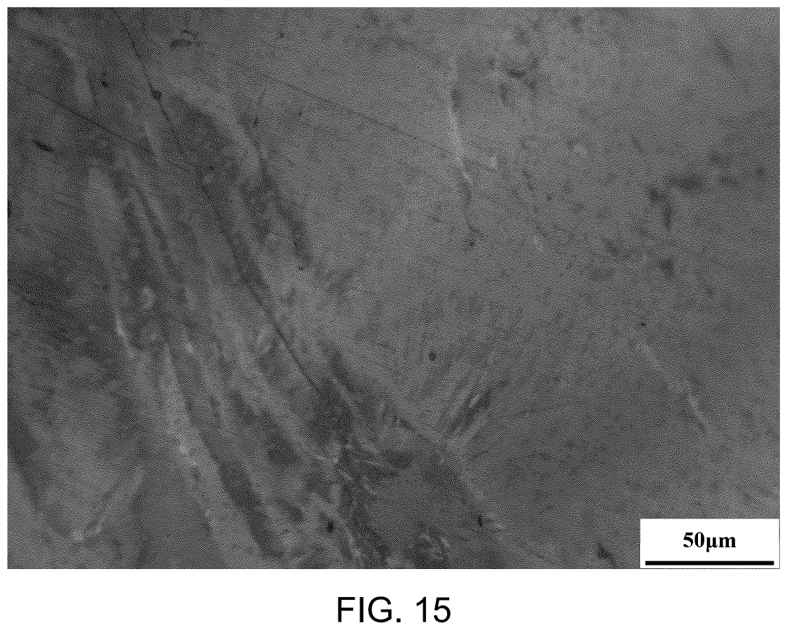

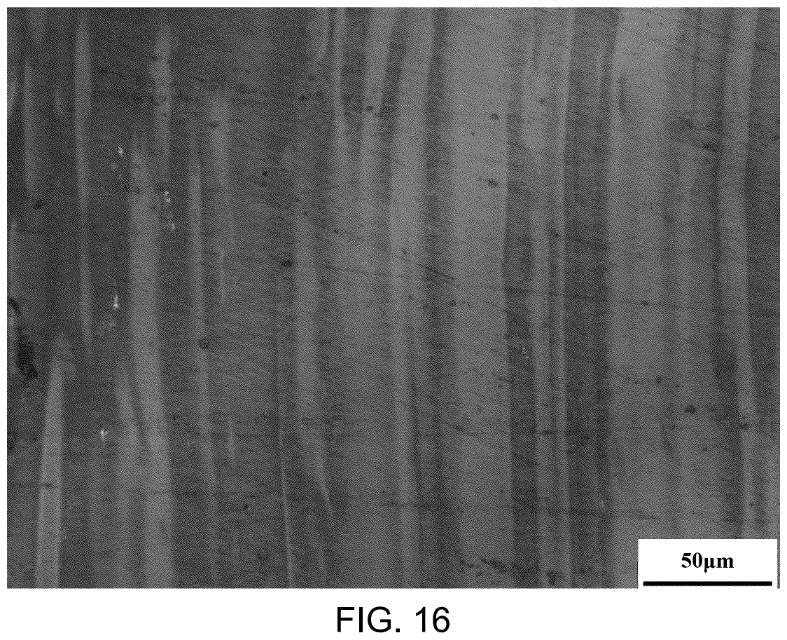

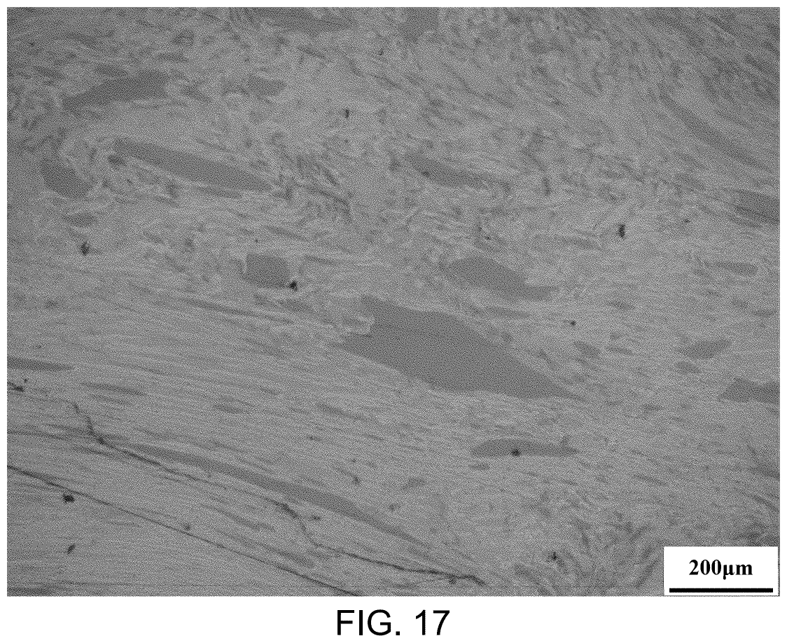

Samples of the prepared mesophase pitch were taken and analyzed for their type and mesophase content using a polarizing microscope respectively. FIG. 14 is a polarized light micrograph of mesophase pitch prepared using the naphthalene oligomer of Example 1 as raw material, FIG. 15 is a polarized light micrograph of mesophase pitch prepared using the naphthalene oligomer of Comparative Example 1 as raw material, FIG. 16 is a polarized light micrograph of mesophase pitch prepared using the naphthalene oligomer of Comparative Example 2 as raw material, and FIG. 17 is a polarized light micrograph of mesophase pitch prepared using the naphthalene oligomer of Comparative Example 3 as raw material. It can be seen from FIG. 14-FIG. 17 that the mesophase pitch prepared using the naphthalene oligomer of Example 1 as raw material is of a wide-area type, with a mesophase content close to 100%; while the mesophase pitch prepared using the naphthalene oligomers of Comparative Examples 1-3 as raw materials is of a streamline type, with a mesophase content of about 97%.

Finally, it should be noted that the above embodiments are only used to illustrate the technical solutions of the present application, but not to limit them; although the present application has been described in detail with reference to the foregoing embodiments, those of ordinary skill in the art should understand that they can still modify the technical solutions described in the foregoing embodiments, or replace some or all of the technical features therein; and these modifications or replacements do not make the essence of the corresponding technical solutions deviate from the scope of the technical solutions of the embodiments of the present application.

What is claimed is:

1. A preparation method of an aromatic hydrocarbon oligomer, wherein the preparation method comprises: subjecting an aromatic hydrocarbon compound to an oligomerization reaction under catalysis of a chloroaluminate ionic liquid to obtain the aromatic hydrocarbon oligomer;
wherein the oligomerization reaction is carried out in a solvent system, and the solvent is selected from chlorinated hydrocarbon solvents;
the aromatic hydrocarbon compound is selected from fused-ring aromatic hydrocarbons with 2 to 4 rings.

2. The preparation method according to claim 1, wherein the chlorinated hydrocarbon solvent is selected from one or more of dichloromethane, trichloromethane, and dichloroethane.

3. The preparation method according to claim 1, wherein the aromatic hydrocarbon compound is selected from one or more of naphthalene, methylnaphthalene, anthracene, phenanthrene, and pyrene.

4. The preparation method according to claim 1, wherein the chloroaluminate ionic liquid has a molecular formula of Et₃NHCl-xAlCl₃, where 1<x≤2.

5. The preparation method according to claim 1, wherein an amount of the chloroaluminate ionic liquid is 5 mol %-25 mol % of an amount of the aromatic hydrocarbon compound.

6. The preparation method according to claim 2, wherein an amount of the chloroaluminate ionic liquid is 5 mol %-25 mol % of an amount of the aromatic hydrocarbon compound.

7. The preparation method according to claim 3, wherein an amount of the chloroaluminate ionic liquid is 5 mol %-25 mol % of an amount of the aromatic hydrocarbon compound.

8. The preparation method according to claim 4, wherein an amount of the chloroaluminate ionic liquid is 5 mol %-25 mol % of an amount of the aromatic hydrocarbon compound.

9. The preparation method according to claim 1, wherein a temperature of the oligomerization reaction is 30-180° C.

10. The preparation method according to claim 1, wherein a time of the oligomerization reaction is 1-11 h.

11. The preparation method according to claim 9, wherein a time of the oligomerization reaction is 1-11 h.

12. The preparation method according to claim 9, wherein the temperature of the oligomerization reaction is 30-90° C., and a time of the oligomerization reaction is 1-3 h.

13. The preparation method according to claim 10, wherein a temperature of the oligomerization reaction is 30-90° C., and the time of the oligomerization reaction is 1-3 h.

14. The preparation method according to claim 11, wherein the temperature of the oligomerization reaction is 30-90° C., and the time of the oligomerization reaction is 1-3 h.

15. The preparation method according to claim 1, wherein a volume ratio of the solvent to the chloroaluminate ionic liquid is (50-500):1.

16. The preparation method according to claim 2, wherein a volume ratio of the solvent to the chloroaluminate ionic liquid is (50-500):1.

17. The preparation method according to claim 1, wherein after completion of the oligomerization reaction, the method further comprises a post-treatment process for a resulting reaction system, the post-treatment process comprising: adding an alkali solution to a reaction solution after the completion of the oligomerization reaction to react with the chloroaluminate ionic liquid in the reaction solution; then standing for layering to collect an organic phase; and subjecting the organic phase to water washing, concentrating, and drying treatments to obtain the aromatic hydrocarbon oligomer.
